(19) United States
(12) Patent Application Publication
(10) Pub. No.: US 2005/0003029 A1
(43) Pub. Date: Jan. 6, 2005

(54) UTILIZATION OF RUTINS AND AESCINS IN THE TREATMENT OF OCULAR CIRCULATORY DISTURBANCES

(76) Inventor: Kailash Kumar Gauri, Hitzhusen (DE)

Correspondence Address: OBLON, SPIVAK, MCCLELLAND, MAIER & NEUSTADT, P.C., 1940 DUKE STREET, ALEXANDRIA, VA, 22314, US

(21) Appl. No.: 10/476,484

(22) PCT Filed: May 10, 2002
(86) PCT No.: PCT/EP02/05178

(30) Foreign Application Priority Data
May 10, 2001 (DE) 101 22 714.0

(51) Int. Cl. A61K 35/78; A61K 31/7048

(52) U.S. Cl. 424/769; 514/27; 514/456

(57) ABSTRACT

The invention relates to the utilization of rutins and aescins in the treatment of ocular circulatory disturbances. The invention also relates to agents with a corresponding active ingredient combination and agents in the form of commercial packagings with corresponding combination preparations or monopreparations for combined application.

The present invention relates to the use of rutins and escins for the treatment of ocular circulatory disturbances. Also described are compositions containing a corresponding active compound combination and compositions in the form of commercial packs containing corresponding combination preparations or mono-preparations for combined administration.

Rutins are glycosides of the flavone quercetin which occur in many plant species. Rutin, which is known under the international nonproprietary name (INN) rutoside, is usually employed in the form of the acidic sodium salts against capillary hemorrhages and further conditions accompanying increased capillary brittleness and membrane permeability. It has therefore often been designated as an “antipermeability factor” or as vitamin P.

Instead of rutin, synthetic rutin derivatives are frequently also used. These include, in particular, O-(β-hydroxyethyl)rutins, which are often obtained as a mixture of rutins which are substituted 1 to 4 times and on different positions of the quercetin by hydroxyethyl groups. An important representative of these derivatives is troxerutin, which can be employed as the main component of a mixture of O-(β-hydroxyethyl)rutins or as a pure substance for the treatment of disorders of the veins and sequelae, in particular in chronic venous insufficiency, varicosis, varicose ulcer and thrombophlebitis. Further indication areas should be ophthalmological applications, such as diabetic retinopathy, retinal and vitreous body hemorrhages, subconjunctival hemorrhages and thromboses.

In the case of escin, a saponin mixture isolable from horse chestnuts, a common venous therapeutic is concerned, which is valued as an escin-containing horse-chestnut seed extract or as a purified escin because of an edemoprotective or antiexudative action.

Accordingly, in the field of venous therapeutics combination preparations are also supplied which contain representatives of both classes of active compound. For instance, horse-chestnut seed extracts containing O-(β-hydroxyethyl)rutins or rutin sulfuric acid esters are recommended in venous circulatory disturbances, such as edema, cramps in the calf, itching, and pain and a feeling of heaviness in the legs, swellings and congestive states caused by varicose veins, varicosis and post-thrombotic syndrome, ulcers of the leg, hemorrhoids, and post-traumatic and post-operative soft-tissue swellings (cf. Rote Liste 2000, Aulendorf: ECV, Editio Cantor Verlag, entries 83044 and 83046).

WO 98/51291 further mentions, inter alia, rutoside, troxerutin or escin for the treatment and prevention of ischemic disturbances.

Ocular circulatory disturbances, in particular if they concern the retina and/or choroid, lead to an often irreversible loss of function of the eyes, i.e. to restricted sight or even blindness.

Retinal hemorrhages, exudates, edemas, ischemias or infarcts are seriously increasing symptoms which need effective treatment. If these symptoms are to be attributed to systemic vascular disorders, for example high blood pressure or diabetes mellitus, the treatment of a corresponding hypertonic or diabetic retinopathy is thus carried out by treating the high blood pressure or diabetes mellitus. However, the therapeutic success achievable in this way, aimed at the symptomatology in the eye, is often not satisfactory. For example, in the syndrome of diabetes mellitus, retinal hemorrhages and retinal edemas often remain which, in spite of normalized blood sugar levels, can worsen further.

Therefore it is frequently necessary, in addition to a treatment of the basic disorder, e.g. a normalization of the blood sugar level, additionally to carry out an antiedematous or circulation-promoting therapy.

The coagulation therapies which are to be carried out for this purpose in the field of ophthalmology, as a rule by means of laser, xenon light or freezing, are, however, disadvantageous because of the scarring caused thereby and the accompanying loss of vision. Even systemic therapies using vessel-sealing agents, such as, for example, calcium dobesilate, or using circulation-promoting agents, such as, for example, pentoxyphylline or pentyphylline, do not offer any satisfactory possibility of treatment for the field of ocular circulatory disturbances.

It has now been found that certain combined uses of rutins and escins open up a surprisingly effective possibility of treatment of ocular circulatory disturbances.

The present invention therefore relates to the use of at least one flavonoid from the group consisting of the rutins, namely of rutin, physiologically acceptable derivatives and/or salts thereof, in combination with at least one saponin from the group consisting of the escins, namely of escin, physiologically acceptable derivatives and/or salts thereof, for the treatment of ocular circulatory disturbances.

The use according to the invention of rutin, physiologically acceptable derivatives or salts thereof—for the purpose of simplification also designated as “rutins” or “rutin component”—and the use of escin, physiologically acceptable derivatives or salts thereof—for the purpose of simplification also designated as “escins” or “escin component”—offers significant advantages in the treatment of ocular circulatory disturbances.

The rutin component and the escin component can in principle be administered together in one formulation or separately in at least two different formulations. The latter possibility comprises both the simultaneous and the temporally separate administration, i.e. taking place at different points in time. A particular embodiment of the temporally separate administration is realized by the alternate administration of both components, for example with an early/late diurnal rhythm.

In this sense, the invention relates to compositions for the treatment of ocular circulatory disorders, which are based on a combination of at least one rutin, of a physiologically acceptable derivative and/or salt thereof and at least one escin, of a physiologically acceptable derivative and/or salt thereof, and, if appropriate, further active compounds, where the active compound components, in particular the rutin component and escin component, can be formulated together or separately.

“Rutin” designates according to the invention 3-[[6-O-(6-deoxy-α-L-mannopyranosyl)-β-D-glucopyranosyl]oxy]-2-(3,4-dihydroxyphenyl)-5,7-dihydroxy-4H-1-benzopyran-4-one, also called quercetin-3-rutinoside or rutoside (INN), of the formula I

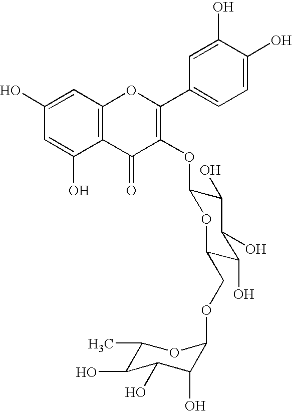

The rutin derivatives especially include O-(β-hydroxyethyl)rutins, in particular the corresponding mono-, bis-, tris- and tetra(hydroxyethyl) derivatives including the respective regioisomeric forms. For example, monoxerutin, i.e. 7-mono-O-(β-hydroxyethyl)rutin and especially troxerutin, i.e. 3′,4′,7-tris-O-(β-hydroxyethyl)rutin of the formula II 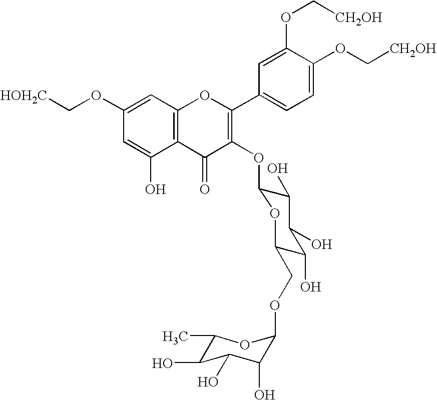
may be mentioned.

Further physiologically acceptable derivatives of rutin include, for example, ethoxazorutin, 8,8′-methylenebis[6-diethylaminomethylrutin], rutin sulfuric acid ester, diosmin (2,3-dehydrohesperidin).

The physiologically acceptable salts of rutin or rutin derivatives in the present case preferably include base addition salts, which are formed in particular with acidic esters, e.g. the sulfuric acid esters, of rutin.

The base addition salts include salts with inorganic bases, for example metal hydroxides or carbonates of alkali metals, alkaline earth metals or transition metals, or with organic bases, for example ammonia or basic amino acids, such as arginine and lysine, amines, e.g. methylamine, dimethylamine, trimethylamine, triethylamine, ethylamine, diethylamine, ethylenediamine, ethanolamine, diethanolamine, 1-amino-2-propanol, 3-amino-1-propanol or hexamethylenetetraamine, saturated cyclic amines having 4 to 6 ring carbon atoms, such as piperidine, piperazine, pyrrolidine and morpholine, and further organic bases, for example N-methylglucamine, creatine and tromethamine, as well as quaternary ammonium compounds, such as tetramethylammonium and the like.

Salts with inorganic bases, e.g. Na, K, Mg, Ca, Zn, Cr and Fe salts, are preferred.

Rutin can be obtained from natural sources, in particular the flower buds of Sophora japonica or buckwheat herbage. For example, the drug material can firstly be extracted with hot water or lower alcohols, the extracts obtained concentrated and optionally defatted using suitable solvents. The crude rutin depositing on cooling can then be recrystallized from water or ethanol or dissolved by addition of alkali and precipitated again using acids.

Moreover, rutin is also accessible synthetically, for example by reacting 7,4′-dibenzylquercetin with hexaacetobromorutinose under suitable conditions, for example coupling in pyridine in the presence of Ag₂CO₃. The acetyl ester groups can then be hydrolyzed and the benzyl protective groups removed, for example hydrogenolytically by means of Pd/C. If necessary, the crude rutin is recrystallized, for example from methanol.

O-(β-Hydroxyethyl)rutins can be obtained by hydroxyethylation of the phenolic groups of rutin with suitable reagents such as 2-chloroethanol or glycochlorohydrin. As a rule, this reaction is carried out in an alkaline medium, for example in the presence of NaOH.

The term “escin” describes a saponin mixture of mainly diacetylated tetra- and pentahydroxy-β-amyrin compounds isolable from horse chestnuts (Aesculus hippocastanum) and in particular from their seeds, which in position 3 carry a glucuronic acid substituted by sugar radicals, for example glucose, galactose and/or xylose. The aglycones are known under the designation barringtogenol of the formula III 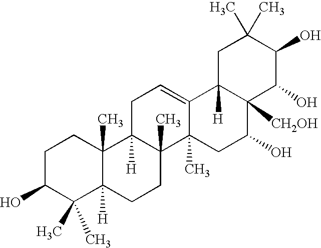
and protoescigenin of the formula IV

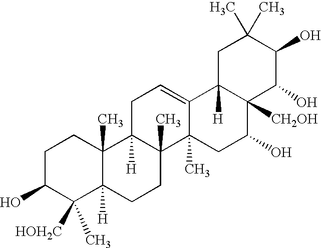

In position 21, different amounts of angelic, tiglic, α-methylbutyric and isobutyric acid are bound in ester-like manner. The term “escin” includes α-escin, β-escin and cryptoescin, which carry acetyl groups in different positions, for example on the 22-α-hydroxyl(β-escin) or on the 28-hydroxyl (cryptoescin). Preferably, the escins are used as a horse chestnut seed extract or in isolated form.

Suitable horse chestnut seed extracts include fluid extracts which are obtainable using alcohol-water mixtures, and dry extracts which can be obtained from the fluid extracts by subsequent drying, preferably spray drying. Suitable extracting agents are, for example, aqueous ethanol or methanol. Good escin yields are obtained, for example, by extraction with 40 to 60% strength ethanol or methanol. In particular, dry extracts (4-8:1) which are standardized on triterpene glycosides, calculated as escin, are common.

Isolated escin can be isolated from horse chestnut seeds, for example, by means of chromatography using ion exchangers (resins).

For further explanation, reference may be made, for example, to EP 0 900 563 A1, which relates to the preparation of escin-containing pharmaceutical preparations.

The physiologically acceptable derivatives of escin in the present case include, for example, esters with preferably organic acids, in particular carboxylic acids, e.g. acetic acid, tartaric acid, lactic acid, citric acid, malic acid, mandelic acid, ascorbic acid, maleic acid, fumaric acid, gluconic acid or sulfonic acids, e.g. methanesulfonic acid, benzenesulfonic acid and toluenesulfonic acid, and the like. Acids of this type are mainly bonded to one or more OH groups in position 21, 22 and also 28. The tartrate, for example, has proven expedient.

In addition to the rutin and escin components, the treatment according to the invention can additionally include further active compounds. These active compounds can be, in particular, those whose action is similar to the rutin- or escin-mediated action or supplements this. Thus it can be advantageous, additionally to the combination according to the invention, to administer ophthalmologicals, venous therapeutics, anti-hemorrhagics and similar active compounds. In particular, it can be expedient to administer antiinflammatory active compounds of the corticoid type, e.g. glucocorticoids, or of the noncorticoid type, such as, for example, indomethacin, or acetylsalicylic acid or derivatives thereof. Likewise, it can be expedient to administer thrombolytic active compounds, such as, for example, streptokinase or urokinase.

A particular embodiment of the present invention is based on the combination of troxerutin with escin or a physiologically acceptable salt thereof.

Ocular circulatory disturbances are understood as meaning circulatory disturbances which affect the eye or parts thereof. These especially include circulatory disturbances of the retina, of the choroid, of the ciliary bodily, of the iris, of the optic nerve, of the sclera, of the cornea. According to a particular embodiment, the present invention therefore relates to the acute or preventive treatment of circulatory disturbances of the eye and of the optic pathway. Occasionally, the circulatory disturbances treatable according to the invention also include those disturbances which can lead to ocular circulatory disturbances without such a condition having to be specified at the time of treatment. These include, for example, inflammatory processes, in particular of the retina (retinitis); of the choroid (choroiditis); of the sclera (scleritis); of the iris (iritis); of the ciliary body (cyclitis); of the cornea (keratitis); of the iris and of the ciliary body (iridocyclitis); of the choroid and of the retina (choroiretinitis, retinochoroiditis); of the choroid, iris, cornea and iris (panuveitis); of the optic nerve (optic neuritis) or of its entry site into the eyeball (optic papillitis); of the optic nerve or its entry site into the eyeball with involvement of the retina, of the choroid, of the sclera, of the iris, of the vitreous body and/or of the cornea. A treatment of such disturbances represents a prevention of ocular circulatory disturbances.

Disturbances treatable according to the invention whose cause are ocular circulatory disturbances especially include disorders which are to be attributed to venous insufficiency, in particular to thrombotic changes of the branch veins or central veins, and/or arterial insufficiency, e.g. teleangiectases. These are especially vascular retinopathies, e.g. a central serosa retinopathy, or choroidopathies, as well as symptomatic disturbances connected therewith and sequelae to be attributed thereto, such as hemorrhagic disturbances, that is vascular leakages and hemorrhages, in particular retinal and choroidal hemorrhages, sclerotic retinal changes, exudates, edema, in particular macular edema, subretinal edema or edema of the head of the optic nerve, glaucoma, in particular neovascular glaucoma or venous-related drainage glaucoma, ischemia, infarcts and trophic changes in particular of the choroid (choroid atrophy), of the optic nerve (optic nerve atrophy) and the restrictions of the sight accompanying them.

Preferred embodiments of the present invention are aimed at the treatment of sclerotic retinal changes; edema of the retina (both intraretinal and subretinal edema); hemorrhages of the retina (both intraretinal and subretinal hemorrhages); hemorrhages of the vitreous body; and choroidal hemorrhages, in particular retinal and choroidal hemorrhages as a result of diabetes mellitus, inflammation, and/or sclerotic vascular processes; retinal and choroidal edema (both intraretinal and subretinal or both choroidal and perichoroidal edema); choroid detachment; serous retinal detachment; hypotonia syndrome, also as a result of vascular processes, in particular sclerotic vascular processes, of vascular leakages, of perfusion disturbances, of diabetes mellitus and/or of inflammation.

Further preferred embodiments of the present invention are aimed at the treatment of trophic disturbances of the optic nerve, of the head of the optic nerve, of the choroid, of the retina, of the sclera, of the ciliary body, of the iris, and/or of the cornea, and further also trophic disturbances in the region of the eyelids and of the lacrimal glands.

The use according to the invention gains in importance in adults with increasing age. In the group consisting of the over 40-year olds and especially the over 50-year olds, the treatment is accompanied by particular advantages. The juvenile diabetics form a further group in which the treatment according to the invention can be accompanied by particular advantages.

Disorders to be treated according to the invention are frequently characterized by progressive development, i.e. the conditions described above change in the course of time, as a rule the degree of severity increases and conditions may change into one another or conditions further to already existing conditions can occur. Preventive therapy can in particular be important if changes in the region of the small vessels (microangiopathies) of other organs or body parts than the eyes are detected. As an example, diabetic changes as a result of microangiopathies may be mentioned, which have led, for example, to intracranial changes, or “background retinopathy”, in which as a result of diabetes mellitus changes to the retina have already occurred.

A particular aspect of a treatment within the meaning according to the invention relates to the treatment of acute or chronic disturbances. The treatment can be accomplished symptomatically, for example as symptom suppression. It can be carried out short term, be accomplished medium term, or it can also be a long-term treatment, for example in the course of a maintenance therapy.

According to the invention, an efficacious amount of rutin component and an efficacious amount of escin component, as a rule formulated corresponding to pharmaceutical, veterinary pharmaceutical or foodstuffs technological practice, is administered to the individual to be treated, preferably a mammal, in particular a human and also an agricultural animal or domestic pet.

The treatment is as a rule carried out by single or repeated daily administration of a suitable dose optionally together or alternatively with other active compounds or active compound-containing preparations, so that an individual to be treated of approximately 75 kg body weight is administered a daily dose of approximately 10 mg to 20 g, preferably of approximately 200 mg to 10 g, advantageously of approximately 900 mg to 5 g and in particular of approximately 2 g to 3 g of rutin component, and of approximately 500 μg to 1 g, preferably of approximately 1 mg to 500 mg and in particular of approximately 5 mg to 200 mg of escin component, on oral administration, and of approximately 10 mg to 20 g of rutin component or approximately 25 μg to 500 mg of escin component on parenteral or alternatively intraocular administration. Independently of the escin dose, the administration of an oral daily dose of more than 1 g, preferably of more than 1.8 g and in particular of more than 2 g of rutin component represents a particularly advantageous aspect of the invention.

Amounts and proportions of active compound relate to the active compound, so that for salts and derivatives an appropriate conversion has to be carried out. An adaptation to the body weight may be necessary.

The invention also relates to the production of compositions for the treatment of an individual, preferably of a mammal, in particular of a human and also of an agricultural animal or domestic pet.

One aspect of the present invention is therefore also compositions comprising
i) at least one flavonoid from the group consisting of the rutins, namely rutin, physiologically acceptable derivatives and/or salts thereof, and
ii) at least one saponin from the group consisting of the escins, namely escin, physiologically acceptable derivatives and/or salts thereof, and
   optionally at least one further active compound and a formulation base.

Compositions according to the invention are therefore based on an active compound combination and optionally a formulation base.

The compositions in particular include pharmaceutical compositions, by which veterinary medicinal compositions are also intended. Ophthalmological compositions represent one particular embodiment.

The active compound combination within the meaning of the invention comprises, as active compound component i), at least one rutin, i.e. rutin, physiologically acceptable derivatives and/or salts thereof. Mixtures of these forms are possible and to be taken into consideration in certain cases. According to a particular embodiment, the active compound component i) consists of an O-(β-hydroxyethyl)rutin mixture, which contains troxerutin as the main component, preferably to at least 50% by weight and in particular to at least 80% by weight. According to a further particular embodiment, the active compound component i) consists essentially of troxerutin. The percentage by weight details are based on the total weight of the active compound component i).

The active compound combination within the meaning of the invention comprises, as active compound component ii), at least one escin, i.e. escin, physiologically acceptable derivatives and/or salts thereof. Mixtures of these forms are possible and to be taken into consideration in certain cases. According to a particular embodiment, the active compound component ii) consists of a horse-chestnut seed extract, which preferably contains approximately 10 to 30% by weight of escin. According to a further particular embodiment, the active compound component ii) consists essentially of escin. The percentage by weight details are based on the total weight of the active compound component ii).

Furthermore, the active compound combination within the meaning of the invention can comprise as active compound component iii) further active compounds, for example the active compounds mentioned above in this connection.

The proportion of the active compound combination in the formulation is greater than a proportion optionally present in natural sources. In this sense, the compositions according to the invention are enriched with respect to the active compound combination. In the case of a pharmaceutical composition, the proportion is as a rule of approximately 1 to 60% by weight, preferably approximately 5 to 35% by weight and in particular approximately 10 to 30% by weight.

Details in % by weight relate, if not stated otherwise, to the total weight of the formulation.

The formulation base of formulations according to the invention contains physiologically acceptable auxiliaries. Physiologically acceptable auxiliaries are those which it is known can be used in the field of pharmacy, foodstuffs technology and related fields, in particular those listed in relevant pharmacopeia (e.g. DAB, Ph. Eur., BP, NF), and also other auxiliaries whose properties do not stand in the way of a physiological application.

Suitable auxiliaries can be: wetting agents; emulsifiers and suspending agents; preservatives; antioxidants; anti-irritants; chelating agents; pan-coating auxiliaries; emulsion stabilizers; film-forming agents; gel-forming agents; odor-masking agents; taste corrigents; resins; hydrocolloids; solvents; solubilizers; neutralizing agents; permeation accelerators; pigments; quaternary ammonium compounds; refatting and superfatting agents; ointment, cream or oil bases; silicone derivatives; spreading aids; stabilizers; sterilizing agents; suppository bases; tablet auxiliaries, such as binders, fillers, lubricants, disintegrants or coatings; propellants; drying agents; opacifiers; thickeners; waxes; plasticizers; white oils. A relevant embodiment is based on expert knowledge, as is shown, for example, in Fiedler, H. P., Lexikon der Hilfsstoffe für Pharmazie, Kosmetik and angrenzende Gebiete [Encyclopedia of excipients for pharmacy, cosmetics and related fields], 4th edition, Aulendorf: ECV-Editio-Kantor-Verlag, 1996.

The sum of active compound component and formulation base is as a rule 100% by weight.

Examples of suitable pharmaceutical formulations are solid pharmaceutical forms, such as powders, granules, tablets, in particular film-coated tablets, pastilles, sachets, cachets, sugar-coated tablets, capsules such as hard and soft gelatin capsules, suppositories or vaginal pharmaceutical forms, semisolid pharmaceutical forms, such as ointments, creams, hydrogels, pastes or patches, and liquid pharmaceutical forms, such as solutions, emulsions, in particular oil-in-water emulsions, suspensions, for example lotions, injection and infusion preparations, eyedrops. Implanted delivery devices can also be used for the administration of active compounds according to the invention. Further, liposomes or microspheres can also be used. Solid pharmaceutical forms and in particular capsules or tablets are preferred.

The formulations can be administered, for example, by the oral, rectal, transdermal, subcutaneous, intravenous, intramuscular, intraocular or intranasal route. Oral administration is preferred.

In the preparation of the compositions, the active compounds are usually mixed or diluted with a suitable auxiliary, in this case also to be designated as an excipient. Excipients can be solid, semisolid or liquid materials, which serve as a vehicle, carrier or medium for the active compound. The admixture of further auxiliaries is carried out if necessary in a manner known per se. Shaping steps, if appropriate in combination with mixing processes, can be carried out, e.g. granulation, compression and the like.

In particular, the active compound components can be formulated together. They can, however, also be separately processed first and then combined in a compartmentalized, e.g. multilayer pharmaceutical form. By this means, possible active compound incompatibilities and different active compound properties, such as bioavailability, stability, solubility and the like, can be taken into account.

The invention likewise relates to corresponding monopreparations in the form of commercial packs, from which the combined use according to the invention is to be inferred.

The present invention is illustrated in greater detail with the aid of the following examples, without being restricted thereto.

### EXAMPLE 1

Pharmaceutical Compositions

a) Soft gelatin capsule containing troxerutin and escin
(troxerutin 450 mg + escin 25 mg)

| | |
|---|---|
| Filling: | |
| troxerutin | 450 mg |
| eschin | 25 mg |
| soybean oil (refined) | 440 mg |
| soybean lecithin (E322) | 50 mg |
| highly disperse silica | 5 mg |
| capsule shell: | |
| gelatine | 303 mg |
| glycerol 85% | 87 mg |
| sorbitol 70% | 77 mg |
| purified water | 52 mg |
| iron oxide pigment Brown 75 (E 172) | 3 mg |

b) Tablet containing troxerutin and escin
(troxerutin 250 mg + escin 13.75 mg)

| | |
|---|---|
| troxerutin | 250 mg |
| escin | 13.75 mg |
| lactose | 127.5 mg |
| magnesium stearate | 5 mg |
| talc | 23.75 mg |
| microcrystalline cellulose | 81 mg |

c) Hard gelatin capsule containing troxerutin and escin
(troxerutin 600 mg + escin 33 mg)

| | |
|---|---|
| Filling: | |
| troxerutin | 600 mg |
| escin | 33 mg |
| lactic acid | 272.5 mg |
| magnesium stearate | 14.5 mg |
| talc | 30 mg |
| alginic acid | 50 mg |

d) Furthermore, tablets or sugar-coated cores prepared according to c) can be provided in a known manner with a film coating which is soluble in the stomach or in the small intestine.

### EXAMPLE 2

Efficacy

### CASE EXAMPLE 1

76-year-old male patient with severe visual impairment.

2 relatively small intraretinal hemorrhages on the retina existed, further 8 vascular leakages stainable with fluorescein sodium and detectable using fluorescence angiography. The finding existed for 4 months with the tendency to worsen. The basic disease present was a generalized sclerotic vascular complaint.

A four-week therapy with 125 mg of escin orally daily was first carried out. The finding did not improve under this. On the contrary a further leakage occurred, so that now 9 leakages were present.

After a two-week therapy break, a four-week therapy with 2250 mg of troxerutin orally daily followed. Under this, after 2 weeks 1 leakage sealed, after 4 weeks a total of 3 leakages. The hemorrhages were unchanged. After a three-week therapy break there were again 8 leakages.

Therapy with the combination of 2250 mg of troxerutin and 125 mg of escin daily orally was carried out. After 1 week 2 leakages were sealed, after 4 weeks 6 leakages were sealed. New leakages were not formed. One hemorrhage was almost completely resorbed after 4 weeks, another half resorbed. New hemorrhages had not occurred.

### CASE EXAMPLE 2

62-year-old female patient with suddenly commencing visual impairment, which had existed for 5 months.

A relatively large intraretinal strip hemorrhage existed as a result of a branch vein thrombosis, further 3 vascular leakages detectable by fluorescence angiography. The basic disease present was latent diabetes mellitus.

A three-week therapy with 125 mg of escin orally daily was carried out. The finding did not improve by means of this.

After a three-week break, a three-week therapy with 2250 mg of troxerutin orally daily was carried out. In the 3rd week, the strip hemorrhage resorbed by 10%. The leakages remained unchanged.

After a three-week therapy break, in which the hemorrhage remained stationary, a therapy with 2250 mg of troxerutin and 125 mg of escin orally daily followed. The hemorrhage resorbed to 90% (measured by the starting size) within 3 weeks and 2 vascular leakages were sealed.

### CASE EXAMPLE 3

69-year-old female patient with severe visual impairment.

1 relatively large and 2 smaller retinal hemorrhages existed; further three vascular leakages which were detectable using fluorescence angiography. The basic disease was a generalized vascular complaint. The finding existed for 5 months with the tendency for increasing worsening.

A three-week therapy with 2250 mg of troxerutin orally daily followed. Under this, in the 3rd week a low-grade beginning of resorption of a small hemorrhage occurred and 1 leakage sealed. A three-week therapy break followed. The hemorrhage in resorption enlarged again, the sealed leakage opened and a further leakage formed.

Therapy with 125 mg of escin orally daily now began. The finding did not worsen. An improvement did not occur.

After a three-week therapy break, therapy with the combination of 2250 mg of troxerutin and 125 mg of escin orally daily was carried out over 3 weeks. Under this, a small hemorrhage resorbed completely, a further small hemorrhage resorbed to 40%, the relatively large hemorrhage to 20%. All leakages were sealed and after 3 weeks no longer detectable by fluorescence angiography.

### CASE EXAMPLE 4

46-year-old female patient with severe visual impairment.

A large subretinal hemorrhage existed, further 2 vascular leakages detectable by fluorescence angiography. A risk factor to be seen was the taking for years of a hormonal anticontraceptive.

A therapy with 125 mg of escin orally daily was carried out. Under this, a slight, not exactly quantifiable tendency for resorption of the hemorrhage occurred.

A therapy break of 3 weeks followed. After this, therapy with 2250 mg of troxerutin orally daily followed. Under this therapy, a resorption of the hemorrhage by approximately 5% occurred and 1 leakage was sealed. After a three-week therapy break, the previously sealed leakage reoccurred. The hemorrhage remained unchanged, i.e. it did not enlarge again.

A three-week therapy with the combination of 2250 mg of troxerutin and 125 mg of escin orally daily now followed.

The hemorrhage resorbed by 30% (measured by the starting size) and both leakages were sealed.

1-20. (Cancelled).

21. A method for the treatment of ocular circulatory disturbances, which comprises
administering at least one rutin in combination with at least one escin to a subject in need thereof.

22. The method as claimed in claim 21, wherein the rutin and the escin are administered together or separately.

23. The method as claimed in claim 21, wherein the rutin is an O-(β-hydroxyethyl)rutin.

24. The method as claimed in claim 23, wherein the O-(β-hydroxyethyl)rutin is troxerutin.

25. The method as claimed in claim 21, wherein the escin is present in the form of a horse-chestnut seed extract.

26. The method as claimed in claim 21, wherein the ocular circulatory disturbances are circulatory disturbances of the eye and of the optic pathway.

27. The method as claimed in claim 26, wherein the circulatory disturbances of the eye and of the optic pathway are circulatory disturbances of the retina, of the choroid, of the sclera, of the iris, of the ciliary body, of the cornea and/or of the optic nerve.

28. The method as claimed in claim 27, wherein the circulatory disturbances are vascular retinopathy or choroidopathy.

29. The method as claimed in claim 21, wherein the ocular circulatory disturbances accompany hemorrhagic disturbances.

30. The method as claimed in claim 29, wherein the ocular circulatory disturbances are retinal hemorrhages.

31. The method as claimed in claim 21, wherein the ocular circulatory disturbances accompany edema.

32. The method as claimed in claim 31, wherein the ocular circulatory disturbances are macular edema, subretinal edema or edema of the head of the optic nerve.

33. The method as claimed in claim 21, wherein the ocular circulatory disturbances accompany glaucoma.

34. The method as claimed in claim 33, wherein the ocular circulatory disturbances are neovascular glaucoma or venous-related drainage glaucoma.

35. The method as claimed in claim 21, wherein the ocular circulatory disturbances accompany atrophic changes.

36. The method as claimed in claim 35, wherein the ocular circulatory disturbances are atrophic changes of the choroid or of the optic nerve.

37. The method as claimed in claim 21, wherein a daily dose of more than 1800 mg of at least one rutin and of approximately 1 mg to 500 mg of at least escin are administered orally to the subject or bioequivalent amounts thereof are administered in another way.

38. A method for the treatment of circulatory disturbances of the eye and the optic pathway, which comprises
administering troxerutin in combination with escin to a subject in need thereof.

39. The method as claimed in claim 38, wherein troxerutin is administered orally at a daily dose of approximately 900 mg to 5000 mg and escin is administered orally at a daily dose of approximately 5 mg to 200 mg.

40. The method as claimed in claim 39, wherein the daily dose of troxerutin is more than 2000 mg.

41. The method as claimed in claim 22, wherein the rutin is an O-(β-hydroxyethyl)rutin.

42. The method as claimed in claim 22, wherein the escin is present in the form of a horse-chestnut seed extract.
